(19) United States
(12) Patent Application Publication
(10) Pub. No.: US 2025/0114236 A1
(43) Pub. Date: Apr. 10, 2025

(54) ERGONOMIC BACK PILLOW, ERGONOMIC LEG SUPPORT, AND ERGONOMICFULL BODY SLEEP SUPPORT SYSTEM

(71) Applicant: Jinghong Chen, Milpitas, CA (US)

(72) Inventor: Jinghong Chen, Milpitas, CA (US)

(73) Assignee: Jinghong Chen, Milpitas, CA (US)

(21) Appl. No.: 18/911,222

(22) Filed: Oct. 9, 2024

Related U.S. Application Data
(60) Provisional application No. 63/543,286, filed on Oct. 9, 2023.

(51) Int. Cl. A61F 5/56

(52) U.S. Cl. CPC A61F 5/56

(57) ABSTRACT

An ergonomic full body sleep support system includes an ergonomic back pillow and an ergonomic leg support for global musculoskeletal alignment and correction. The ergonomic back pillow supporting a global neutral spinal pelvic alignment of the head, neck, torso, and hip, consists of three segments including a transverse head and upper cervical supporting pillow, a transverse lumbar supporting segment, a longitudinal lower cervical and thoracic supporting segment aligned to the longitudinal centerlines of the other two segments, forming a longitudinally symmetrical assembly, a left cut-out and right cut-out to support a range of sleeping positions from supine to lateral sleep. An ergonomic leg support to elevate and facilitate leg venous return consists of a transverse lower portion, longitudinal upper left and right wings, and a cut-out. Ergonomic full body sleep support system provides a pragmatic solution to accommodate global neutral musculoskeletal alignment, facilitate the associated biological functions and sleep comfort.

### FIELD OF THE DISCLOSURE

The present invention relates to a sleep support solution, more specifically relates to an ergonomic neutral global musculoskeletal sleep support solution for improved sleep health and correction of the existing musculoskeletal disorders during sleep.

### BACKGROUND

An average person spends one third of life sleeping. Sleeping appears to be the body's simple way of rest, yet complexed biological processes are underway to recuperate and regenerate the skeletal, muscular, neurological, cardiovascular, immune, and other biological systems. Proper sleep support is important not only for the physical comfort and quality of sleep, but also for maintaining the neutral musculoskeletal structure, biological functions at night, and the overall sleep health.

The pillow and bed have major impact on the skeletal structure that supports the human body. Most people are aware that poor daytime posture can lead to spinal problems, causing muscle and nerve pain in the neck, shoulder, and lower back, and these spinal problems are more pronounced since the computer and cell phone become ubiquitous in the last two decades. However, less people are aware that some of the poor postures developed during sleep while we are unconscious are equally if not more harmful.

Many commercial ergonomic products are designed to address certain aspects of the musculoskeletal alignment problems, such as cervical pillows, back supports, and leg elevations. There are intellectual properties of full-dynamic sleep solutions to address the global musculoskeletal alignment, however in practice only semi-dynamic solutions have been reduced to practice such as inflatable or adjustable pillows and mattresses, which have achieved limited commercial success due to many practical challenges such as the high price, discomfort associated with the materials in inflatable products, and the adjustability only in large sections for the supine sleeping position in the adjustable mattresses with difficulty to accommodate both supine and lateral sleeping positions effectively.

### SUMMARY

The present invention presents an ergonomic full body sleep support system comprising an ergonomic back pillow that enables the neutral spinal pelvic alignment of the head, neck, torso and hip, and an ergonomic leg support that elevates the legs and facilitates their venous return.

The innovative ergonomic back pillow is composed of three segments of a transverse head and upper cervical supporting pillow, a longitudinal lower cervical and thoracic supporting segment, and a transverse lumbar supporting segment, wherein the longitudinal centerline of the longitudinal lower cervical and thoracic supporting segment is aligned respectively to the longitudinal centerlines of the transverse head and upper cervical supporting pillow and the transverse lumbar supporting segment, forming a longitudinally symmetrical assembly, as well as a left cut-out and a right cut-out surrounded by the three segments for placing the shoulder and arms, and for rolling from the supine to the lateral sleeping position. In addition to the above single-form, the ergonomic back pillow can also be in a side-by-side plurality-form for multiple people.

The longitudinal center portions of the ergonomic back pillow include the longitudinal center portion of the transverse head and upper cervical supporting pillow, the entirety of the longitudinal lower cervical and thoracic supporting segment, and the longitudinal center portion of the transverse lumbar supporting segment, forming an ergonomic supine sleep entity with proper dimension and profile, when compressed under the body weight, provides the neutral spinal pelvic alignment, an elevated head relative to the heart, a mandible-cervical angle for an open airway and the minimal snoring, an open shoulder and chest for the efficient respiration and blood circulation, and a uniform contact pressure on the body parts for more comfort in the supine sleeping position.

The longitudinal left and right portions of the ergonomic back pillow include the longitudinal left and right portions of the transverse head and upper cervical supporting pillow, and the longitudinal left and right portions of the transverse lumbar supporting segment, forming an ergonomic lateral sleep entity with proper dimension and profile, when compressed under the body weight, provide the neutral spinal pelvic alignment, an elevated head relative to the heart, an open shoulder and chest for the efficient respiration and blood circulation, and a uniform contact pressure on the body parts for more comfort in the lateral sleeping positions.

The longitudinal lower cervical and thoracic supporting segment is constrained in its width such that the left and right cut-outs are sufficient for the shoulder and arms to roll from the supine to the lateral sleeping positions, and its top and side profiles provide back support for a range of sleeping positions from the supine to the lateral sleep. The overall profile of the ergonomic back pillow has a gradual and smooth transition from the supine sleep entity to the lateral sleep entity to avoid abrupt change of the contact pressure, to accommodate the natural transition, and to stabilize a range of sleeping positions from the supine to the lateral sleep, thus reducing the probability of the supine sleep and snoring, reducing the probability of prolonged pressure on any body parts in one particular sleeping position, and minimizing the arousal and providing better sleep comfort.

The ergonomic back pillow is in form of a single unit containing the three segments, or discrete units containing one or two of the three segments. The single unit or discrete units can be enclosed in a single fabric cover forming an ergonomic back pillow unit; the discrete units can be attached to each other by fabric fasteners forming an ergonomic back pillow assembly, or attached to a thin mat by fabric fasteners forming an ergonomic back pillow pad assembly. The single unit and each unit of the discrete unit can be further divided forming compartments. The compartment of the single unit or discrete units can have one layer or multiple layers of the stuffing materials.

The ergonomic back pillow offers structural flexibility to adapt to people with different height and body types, and to satisfy the corrective needs of patients with various degrees of different types of musculoskeletal disorders. In fact, the ergonomic back pillow, by leveraging the long hours of sleep, could be a more practical and effective passive correction in resolving the musculoskeletal disorders than the active approaches of day time physical therapies or corrective exercises, simply because it is the only way most of the people can afford to stay in one corrective position for a long period of time. The ergonomic back pillow is adjustable: in the longitudinal length by controlling the gap length between the adjacent discrete units, for example, the ergonomic back pillow unit having three discrete units is enclosed in a single cover composed of three respective compartments with a zip extension compartment at each end of the lower cervical and thoracic supporting segment, and its longitudinal length can be adjusted by unzipping the zip extension compartments, and for another example, the longitudinal length of the ergonomic back pillow pad assembly can be adjusted by controlling the gap length between the adjacent discrete units; in the height and profile by adding different number of layers of the stuffing materials with the same or different thickness under the top profile layer in each compartment of the single unit or discrete units, for example, the head and upper cervical supporting pillow can have two alternative top profiles in the upper and lower transverse halves for people with different preference, and for another example, the head and upper cervical supporting pillow can be divided into the center supine sleep compartment and two side lateral sleep compartments each with longitudinal upper and lower halves, the lower cervical and thoracic supporting segment can be divided into proximal and distal compartments, and the lumbar supporting segment can be divided into the center supine sleep compartment and two side lateral sleep compartments, and the height and profile of each compartment can be adjusted according to the above-mentioned method.

In one embodiment, the invented ergonomic back pillow is a single unit with the above mentioned three segments. The transverse head and upper cervical supporting pillow is roughly in a cuboid shape with the top surface recessed along the centerlines of both the transverse and longitudinal directions, and its dimensions and profiles of longitudinal center, left and right, when compressed under the body weight, support the neutral head and upper cervical vertebra in the supine, left and right lateral sleeping positions respectively, allowing good blood circulation in the head and less snoring. The longitudinal lower cervical and thoracic supporting segment is roughly in a half cylindrical shape with proper dimension and profile, when compressed under the body weight, matching the back profile within the scapular distance for the supine sleeping position, providing back support for a range of sleeping positions from the supine to the lateral sleep, accommodating the neutral cervical thoracic curvatures, and allowing the shoulder and chest to fully open for better respiration and blood circulation. The transverse lumbar supporting segment is also roughly in a half cylindrical shape with its longitudinal width matching the lumbar pelvic span, and its dimensions and profiles of the longitudinal center, left and right, when compressed under the body weight, support the neutral lumbar pelvic curvatures in the supine, left and right lateral sleeping positions respectively. The height and profile are adjustable by adding the stuffing layers with the same or different thickness under the top profile layer in each compartment of the single unit.

In another embodiment, the invented ergonomic back pillow has the same ergonomic dimension and profile as the above embodiment except that the three segments are in three discrete units, either enclosed in a single cover composed of three respective compartments with a zip extension compartment at each end of the lower cervical and thoracic supporting segment, forming an ergonomic back pillow unit; or enclosed in three separate covers attached to each other by fabric fasteners on their surfaces, forming an ergonomic back pillow assembly; or enclosed in three separate covers each with Velcro strips on the bottom surfaces attached to a thin mat with matching Velcro strips on the top surface, forming an ergonomic back pillow pad assembly with adjustable gap length between the adjacent discrete units. The height and profile are adjustable by adding different number of layers of the stuffing materials with the same or different thickness under the top profile layer in each compartment of the discrete units.

Sleep comfort comes from reducing the contact pressure, as well as the management of thermal, moisture, and air permeability by the stuffing and cover materials. The stuffing materials also dictate product firmness and conformability. The invented ergonomic back pillow is in a form of a single unit or discrete units with further divided compartments each having one layer or multiple layers with the same or different stuffing materials composed of synthetic materials such as foam, memory foam, shredded foam, reticulated foam, 3D air-weave polymer fiber network, latex, synthetic fibers, and cooling micro-beads or cooling micro-gel infiltrated foam; plant based materials such as cotton, kapok, flax, hemp, ramie, jute, sisal, coir, reed, luffa sponge, Rayon, Modal, Viscose, Lyocell, buckwheat hull, millet hull, dried leaves, and straws; animal based materials such as wool, silk, down, and feather; metal containing materials such as metal fiber or weave, metal coated or plated fiber or weave, metal ribbon or metal coated film ribbon wrapped fiber or weave, metal coated or plated 3D structures of luffa or reticulated foam or air-weave polymer fiber network, metal oxide or metal salt infused fiber or weave, and metal, metal oxide, or metal salt infiltrated foam; and any of their combinations. Synthetic memory foam is a popular stuffing materials choice due to the ease of ergonomic shape forming and the versatility of property selection. Additional design features are built into synthetic memory foam to further improve its thermal management and air permeability, such as arrays of vertical through-holes distributed across the memory foam, grooves and/or grids on the surface and/or inside the memory foam, large vertical through void at certain locations, and any of their combinations. Plant and animal based materials are used as stuffing materials due to their excellent thermal and moisture management, and air permeability.

In one embodiment, the ergonomic back pillow is made of open-cell polyurethane memory foam due to wide selection of firmness, ease of forming ergonomic shapes, conformability to human body, and decent air permeability. Arrays of small vertical through-holes are distributed across the memory foam, square or honeycomb patterned grooves are on the top and bottom surfaces of the memory foam, three large vertical through holes are in the pillow at the centers of the supine and lateral sleeping positions.

In anther embodiment, the ergonomic back pillow is made of a three-layer stuffing materials comprising a bottom layer of the above open-cell polyurethane memory foam with the above design features to provide the ergonomic shape and support, a middle layer of a thin pad comprising highly breathable materials such as flax fiber or buckwheat hull, and a top layer of cotton thin pad for the softness and sweat wicking.

In another embodiment, the head and lower cervical supporting pillow of the ergonomic back pillow is made of a multi-layer stuffing materials for reduced head temperature, comprising a bottom layer of the above open-cell polyurethane memory foam with the above design features, a middle layer of a thin pad comprising highly breathable materials such as flax fiber or buckwheat hull, a top layer of a thin pad comprising copper fiber weave for better thermal management.

The fabric covers of the ergonomic back pillow and thin mat are in direct contact with human body, its material selection and weave pattern have profound impact on the thermal and moisture management, air permeability, and sleep comfort. The fabric cover is in the form of a single unit or discrete units with further divided compartments, in one layer or multiple layers composed of the same or different materials of plant fibers, animal fibers, synthetic fibers, metal containing or metal infused fibers, and any of their combinations. The fiber microstructures and weave patterns have different wicking ability and porosity, thus providing different thermal and moisture management, and air permeability. The fabric cover has features for opening and cleaning, and fabric fasteners to attach to other parts.

In one embodiment, the fabric cover of the ergonomic back pillow has a top layer of weaved cotton with good wicking ability and sleep comfort, and a bottom layer of weaved jute with high porosity for improved thermal and moisture management, and air permeability.

In another embodiment, the fabric cover of the ergonomic back pillow has a top layer of weaved fabric made of cotton and silver or Au coated thread, providing good wicking ability and improved heat dissipation, and a bottom layer of fabric made of copper containing or copper infused fiber and polyester for improved thermal management.

The elevation of the feet is traditionally accomplished with an elevation wedge, a pillow, or a multi-zone mattress with higher firmness in the lower limb section. In the present invention, the ergonomic leg support is an economic approach to elevate the lower limbs working together with the ergonomic back pillow to provide neutral skeletal support to the whole body. It has a transverse lower portion to elevate the legs when lying straight in the supine and lateral sleeping positions, a longitudinal upper left wing and a longitudinal upper right wing above the transverse lower portion for the elevation and side support of the legs to bend up sideway and anchor in the lateral sleeping positions, and a cut-out surrounded by the transverse lower portion and the two wings for the hip and upper thighs. Comparing to anchoring the body linearly in the lateral sleeping position, having one leg bent up at various bending angles provides the body a more stable support on triangular surfaces to distribute body weight and heat. In the absence of the elevation from the ergonomic leg support, however, lying with a leg bent up sideway could twist the spine and pelvic, causing scoliosis and pelvic tilt in a long run. In addition to the above single-form, the ergonomic leg support can also be in a side-by-side fused plurality-form for multiple people.

The invented ergonomic leg support can be in a form of a stand alone unit as an elevation wedge composed of one layer or multiple layers of the stuffing materials each with the same or different thickness; a part of an ergonomic leg elevation mattress comprising or merging the stand alone unit on the top or at the bottom of a mattress; or a part of an ergonomic leg elevation mattress assembly comprising a bottom support layer, an optional middle transition layer, and a top ergonomic leg elevation mattress.

The invented ergonomic leg support offers flexibility to adapt to people with different height and body types. The height of the ergonomic leg support is adjustable by stacking one layer or multiple layers of the stuffing material with the same or different thickness; the position of the ergonomic leg support is adjustable by moving the stand alone unit along the longitudinal direction of the mattress in the ergonomic leg elevation mattress or extending the two wings longitudinally with fitting pads; and the size and height of the cut-out is adjustable by filling the cut out with fitting pads in the longitudinal direction.

Each component of the ergonomic leg support and the fitting pad contain the same or different stuffing materials composed of innerspring; synthetic materials such as foam, memory foam, shredded foam, reticulated foam, 3D air-weave polymer fiber network, latex, synthetic fibers, and cooling micro-beads or cooling-gel infiltrated foam; plant based materials such as cotton, kapok, flax, hemp, ramie, jute, sisal, coir, reed, and luffa sponge; animal-based materials such as wool and hair; metal containing materials of metal coated or plated 3D structures of luffa or reticulated foam or air-weave polymer fiber network, and metal or metal oxide or metal salt infiltrated foam; and any of their combination. Additional features are built into the top foam to further improve its thermal management and air permeability, including arrays of vertical through-holes, grooves and/or grids on the surface and/or in the internal, and any of their combinations.

The stuffing materials of the ergonomic leg support is enclosed in a fabric cover: in the form of a single piece, or a piece with multiple compartments for the ease of the adjustment for the transverse lower portion, the longitudinal upper left wing, the longitudinal upper right wing, and the fitting pad extensions; composed of the same or different materials of plant-based fiber, animal-based fiber, synthetic fiber, metal fiber, metal containing fiber, metal infused fiber, and any of their combinations; contains features for the opening and cleaning.

In one embodiment, the ergonomic leg support stand alone unit is made of open cell polyurethane memory foam with a cuboid transverse lower portion, a cuboid longitudinal upper left and upper right wings and a cuboid cut-out surrounded by the lower portion and two wings. The ergonomic leg support stand alone unit is placed on top of the lower half of an open cell polyurethane memory foam mattress, forming an ergonomic leg elevation mattress. The ergonomic leg support stand alone unit has a width the same as that of the mattress, a flat top surface, an upper peripheral descending gradually to the mattress, and a height to elevate the legs to the level of the heart reaching the zero-gravity position. There are vertical through-holes and groove patterns evenly distributed across the top surface of the ergonomic leg elevation mattress for thermal management and air-permeability. The ergonomic leg support stand alone unit and ergonomic leg elevation mattress are enclosed respectively in an one-piece fabric cover and a two-layer one-piece fabric cover with the top surfaces made of weave of cotton mixed with silver or gold coated fiber.

In another embodiment, the ergonomic leg support stand alone unit is the same as the above embodiment, except that it is inserted underneath the lower half of the above open cell polyurethane memory foam mattress, forming an ergonomic leg elevation mattress. In the absence of the leg compression, the mattress dominants the overall top profile, inducing a minimal topography change with a shallow concave at the cut-out and a gradual decline at the upper peripheral of the ergonomic leg support stand alone unit in comparison to the topography of a traditional mattress. Under the leg compression, the ergonomic leg elevation mattress forms a gentle supporting profile around the legs without any sharp transition. The ergonomic leg elevation mattress is enclosed in a two-layer one-piece fabric cover with the top layer for the mattress and the bottom layer with different compartments for the transverse lower portion, the longitudinal upper left and right wings, the fitting pad extensions respectively in the ergonomic leg support stand alone unit with adjustability in the height, position, wing size, and cut out size.

In another embodiment, the ergonomic leg support stand alone unit is part of a multi-layer ergonomic leg elevation mattress assembly comprising a firm bottom support layer of an innerspring, a lower middle layer of a medium firmness open cell polyurethane memory foam, an upper middle layer of the ergonomic leg elevation mattress in the above embodiment, and a top mattress made of highly breathable fabric materials. The multi-layer ergonomic leg elevation mattress assembly is enclosed in a multi-layer fabric cover with zippers on each layer.

The invented ergonomic full body sleep support system is composed of an ergonomic back pillow and an ergonomic leg support, providing the ergonomic supports for the supine, lateral, and a range of sleeping positions between the supine and lateral sleeping positions with a top profile for gradual transitions between different sleeping positions, and a built-in design flexibility for people with different height and body types. It accommodates the neutral full body musculoskeletal alignments, opens up the shoulder and chest to maximize the respiratory and cardiovascular functions, reduces the chance of heavy snoring, increases the contact area for a reduced average pressure and pressure variation, facilitates the venous return in the lower limbs and head, uses materials and design patterns to better skin health, thus improves the overall physical comfort and sleep quality. In addition, by effectively leveraging the long sleeping time, the invented ergonomic full body sleep support system also serves as a pragmatic corrective device for the existing musculoskeletal disorders.

While multiple embodiments are disclosed, still other embodiments of the present invention will become apparent to those skilled in the art from the following Detailed Description. As will be realized, the embodiments are capable of modifications in various aspects, all without departing from the spirit and scope of the embodiments. Accordingly, the drawings and detailed description are to be regarded as illustrative in nature and not restrictive.

### BRIEF DESCRIPTION OF THE DRAWINGS

The present invention is further detailed with respect to the following drawings. These figures are not intended to limit the scope of the present invention but rather illustrate certain attributes thereof.

FIG. 1A and 1B are the side and back views of a neutral human spine respectively.

FIG. 2A and 2B are the side views of a human body with a neutral spine alignment in the supine and lateral sleeping positions respectively. The vectors F1 to F6 and f1 to f6 are the relative forces, represented by the arrow scales, that are experienced by the corresponding body parts of a human body lying on a uniform flat mattress in the supine and lateral sleeping positions respectively.

FIG. 3A and 3B are the side views of a human body with a neutral spine alignment in the supine and lateral sleeping positions respectively after adding supports under the neck, lumbar, and knees. The vectors F′1 to F′9 and f′1 to f′9 are the relative forces, represented by the arrow scales, that are experienced by the corresponding body parts of a human body lying on a uniform flat mattress in the supine and lateral sleeping positions respectively.

FIG. 4 is a front perspective view of an embodiment of an ergonomic back pillow.

FIG. 5 is a side perspective view of an embodiment of an ergonomic back pillow.

FIG. 6 is a side view of an embodiment of an ergonomic back pillow.

FIG. 7 is a back view of an embodiment of an ergonomic back pillow assembly composed of three discrete segments, each with Velcro strips on its bottom cover to attach to the corresponding Velcro strips on the top cover of a thin mat.

FIG. 8 is a front perspective view of a thin mat with matching Velcro strips on its top cover in an embodiment of an ergonomic back pillow pad assembly.

FIG. 9A, 9B, 9C are the top views of three exemplary ventilation patterns of square patterned grooves with vertical through holes, honeycomb patterned grooves with vertical through holes, and three large vertical through voids respectively on the memory foam of the transverse head and upper cervical supporting pillow in an embodiment of the invented ergonomic back pillow.

FIG. 10 is a side perspective view of an embodiment of a multi-layer ergonomic leg elevation mattress assembly with an ergonomic leg support on the top surface.

FIG. 11 is a side perspective view of another embodiment of a multi-layer ergonomic leg elevation mattress assembly with an ergonomic leg support inserted under the top layer.

FIG. 12A and 12B are the side views of a human body with a neutral spine alignment lying in the respective supine and lateral sleeping positions on an ergonomic full body sleep support system.

FIG. 13 is a top view of a human body with a neutral spine alignment lying in the lateral sleeping position with a leg bent up sideway and anchored on the upper left wing of an ergonomic full body sleep support system.

### DETAILED DESCRIPTION

In the following description of examples, reference is made to the accompanying drawings which form a part hereof, and in which it is shown by way of illustrating specific examples that can be practiced. It is to be understood that other examples can be used and structural changes can be made without departing from the scope of the disclosed examples.

The intervertebral disc (IVD) is the largest avascular structure in the human body. The nutrient supply and metabolic waste disposal in the IVD are through the diffusion and convection from the nearby peripheral blood vessels, and this process follows the diurnal cycle. During the day time, the IVD expels a large amount of disc's fluid under the compressive loads. This reduces the IVD height and increases the osmotic force, as a result, the nutrition and waste transport rate to and from the disc centre respectively are increased. During the night rest, the IVD is decompressed, and the expelled disc's fluid is to be fully imbibed. Poor sleep postures other than a neutral spinal alignment is disruptive to the imbibition of the disc's fluid, the nutrient supply, and metabolic waste disposal, thus causing the IVD cell death and a various spinal degeneration and deformation, and the same mechanism is applicable to the health of other joints. Overtime, this can also lead to lordosis, kyphosis, scoliosis, and pelvic tilt. Poor posture induced human skeletal problems in turn impact the connecting tissues and internal organs, leading to the muscle and nerve pain, and biological functional disorders. Therefore, the pillow and bed should carry the primary goal of providing the ergonomic support to enable the neutral musculoskeletal structure.

Aside from providing the skeletal support, the pillow and bed have intricate influence on many biological functions, most directly on the respiratory and cardiovascular functions. Snoring occurs when the upper airway is narrowed by the surrounding nasal and throat tissues, causing a turbulent airflow that vibrates the tissues and produces noise. Since the muscle tension is released during sleep, snoring occurs more easily, particularly in the supine sleeping position in comparison to the lateral sleeping position as the gravity is pulling some of the tissues towards the airway. Heavy snoring can cause obstructive sleep apnea (OSA), which can lead to serious medical conditions over time. Research indicates that the mandible-cervical angle at the cervical vertebra No. 7 of larger than 55° can increase the planar area of the pharynx in the supine sleeping position, preventing airway collapse and eliminating snoring completely. Therefore, the proper design of the head to cervical angle in the supine sleep and methods to promote the lateral sleep while we are unconscious at night are important factors to reduce and eliminate snoring in the design of an ergonomic support system. The lateral sleep is also proven to be superior relative to the supine sleep in terms of facilitating the glymphatic clearance in the central nervous system (CNS) of the brain, thus have profound impact on the brain diseases such as Alzheimer, multiple sclerosis, and brain tumors. In addition, our body weight is concentrated in the middle, thus a conventional mattress with an uniform firmness turns to depress more in the middle under the body weight, thus compressing the shoulder and chest, and adding burden to the respiratory and cardiovascular systems. Back support features that can open up the shoulder and chest are beneficial for the respiration and blood circulation. Further more, since the blood pressure is significantly lower in sleep than that of awake, and the blood venous circulation is mainly driven by the gravity in sleep, thus elevation of the head and legs to the level of the heart, reaching a so called zero-gravity position, can promote the venous return to the heart, preventing the head and leg edema, and improving sleep quality. It is clear that a full body ergonomic sleep support system is beneficial to proper biological functions and long term health.

The pillow and bed also need to provide comfort for a good night sleep, which means sufficient hours of sleep, and a healthy balance of non-rapid eye movement (NREM) and rapid eye movement (REM) sleep. Human skin is a sensing organ ladened with pressure and thermal receptors that respond to the external stimuli and send signals to the brain. A stimulus above certain threshold for a prolonged period of time can be uncomfortable and cause arousal, leading to less NREM time, more sleep fragmentation, or even complete awakening.

When lying in both the supine and lateral sleeping positions on a conventional mattress with a flat surface and uniform firmness, the localized pressures on the shoulder and hip areas are much higher than those on the other areas of the body. If a part of the body is under a high contact pressure, the blood supply to that area is hindered, causing arousal. For patients lying in bed for a prolonged period of time, this can eventually lead to pressure sores. A typical solution is to add body supports to other areas, minimize the amount and magnitude of the stimuli, and redistribute the pressure evenly throughout the body.

Our body's core temperature naturally drops during sleep, a cool but not cold head temperature is ideal for a good night sleep. Increase in temperature may cause the body to feel warm and sweat at night. In addition, the air permeability is vital for the skin health because the cells in the outer skin layers of our body are almost exclusively supplied by the atmosphere oxygen. The thermophysilogical comfort during sleep is associated with the ambient temperature, humidity, and air circulation, as well as the management of the thermal, moisture, and air permeability by the materials of the sleep support system, thus the selection and design of the stuffing and cover materials in a sleep support system are also important.

There are three common sleeping positions, namely the supine, lateral, and prone positions. Among them, the supine and lateral sleeping positions are the two most adopted positions. People on average switch sleeping positions multiple times throughout a night. The side views of a neutral spine alignment in the supine and lateral sleeping positions corresponding to the respective sagittal and coronal spinal curvatures are different, and human body profile and weight distribution are intrinsically different in the supine and lateral sleeping positions, thus different ergonomic supporting profiles are required to maintain the neutral spinal pelvic curvatures for different sleeping positions.

There is a need of a practical sleep support solution that can accommodate the ergonomic requirements in the common supine and lateral sleeping positions simultaneously. An innovative and cost effective ergonomic full body sleep support solution that provides the neutral global musculoskeletal alignments for both the supine and lateral sleeping positions, facilitates the associated biological functions, and provides the necessary physical comfort is highly desirable and will bring positive contribution to the overall health.

The present invention presents an ergonomic full body sleep support system, comprising an ergonomic back pillow that enables the neutral spinal pelvic alignment of the head, neck, torso and hip, and an ergonomic leg support that elevates the legs and facilitates their venous return. The ergonomic full body sleep support system provides a practical solution to accommodate the neutral global musculoskeletal alignment, facilitates the biological functions, offers adjustability to people with different height and body types, produces the physical comfort for improved sleep quality, and serves as a pragmatic corrective device for the existing musculoskeletal disorders.

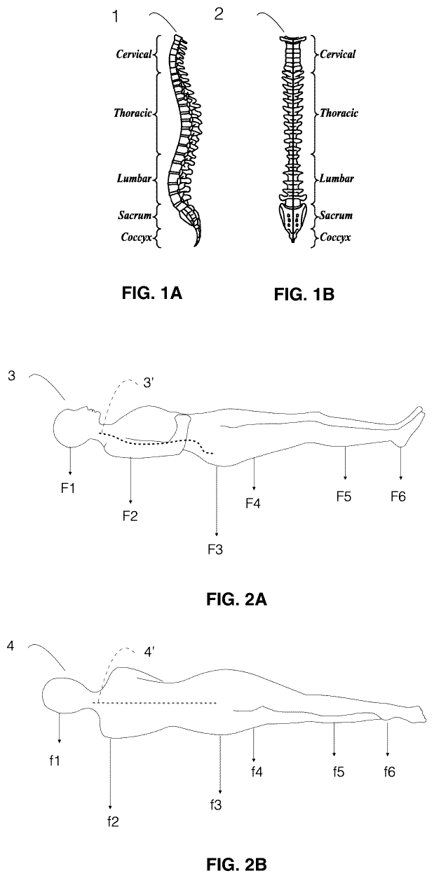

FIG. 1A and 1B illustrate the side view 1 and back view 2 of a neutral human spine in the Sagittal plane and Coronal plane respectively.

FIG. 2A shows the side view of a human body 3 with a neutral spine alignment 3′ in the supine sleeping position, wherein 3′ as shown in the dash line corresponds to the side view 1 illustrated in FIG. 1A. FIG. 2B shows the side view of a human body 4 with a neutral spine position 4′ in the lateral sleeping position, wherein the 4′ as shown in the dash line corresponds to the back view 2 illustrated in FIG. 1B. The vectors F1 to F6 and f1 to f6 are the relative forces, represented by the arrow scales, that are experienced by the corresponding body parts of a human body lying on a uniform flat mattress in the supine and lateral sleeping positions respectively. In both positions, the pressures on the shoulder and hip are higher than those on the other body parts. Therefore there is a need to strategically add supports to allow even pressure distribution, reduce the necessity to keep changing the sleeping positions, thus improve the sleep comfort.

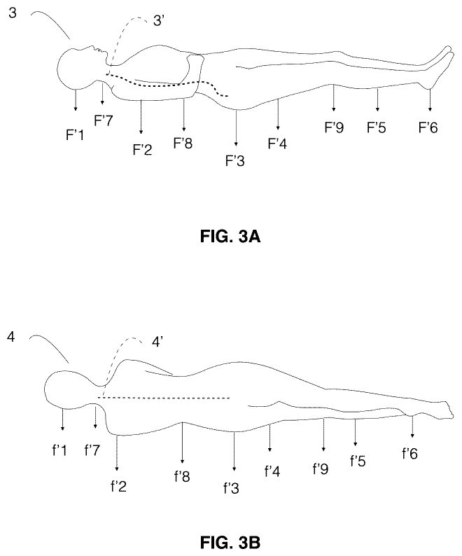

FIG. 3A and 3B are the side views of a human body 3 and 4 with a neutral spine alignment 3′ and 4′, illustrated in the dash lines, in the supine and lateral sleeping positions respectively after adding supports of F′7-F′9 and f′7-f′9 under the neck, lumbar, and knee. The vectors F′1 to F′9 and f′1 to f′9 are the relative forces, represented by the arrow scales, that are experienced by the corresponding body parts of a human body lying on a uniform flat mattress in the supine and lateral sleeping positions respectively. In both cases, the relative forces are more evenly distributed than those in FIG. 2A and FIG. 2B respectively.

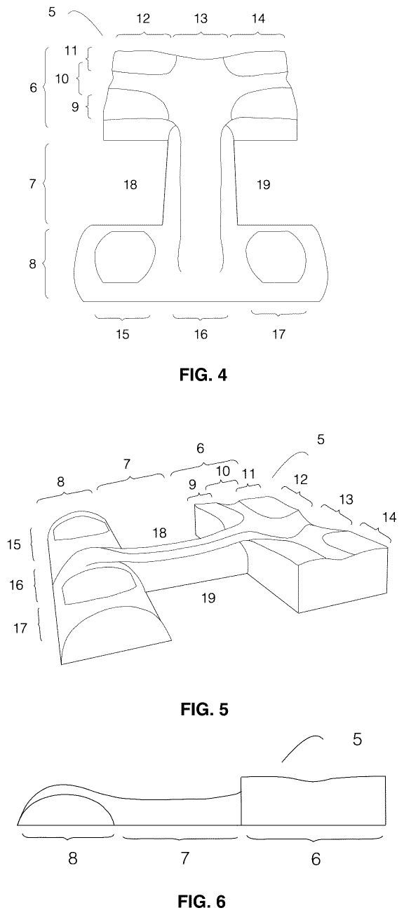

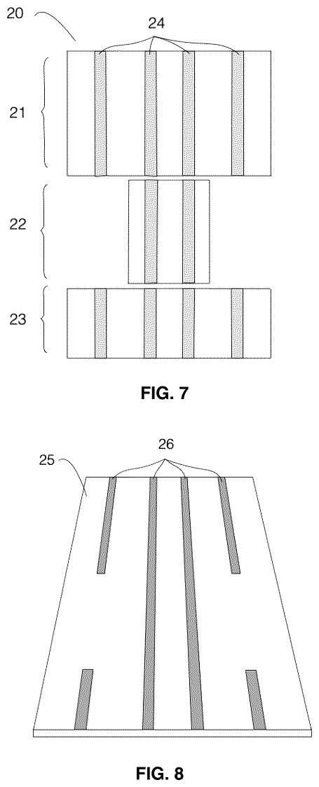

FIG. 4 to FIG. 6 illustrate a front perspective view, side perspective view, and side view respectively of an embodiment of an ergonomic back pillow 5. The ergonomic back pillow 5 has three segments composed of a transverse head and upper cervical supporting pillow 6, a longitudinal lower cervical and thoracic supporting segment 7, and a transverse lumbar supporting segment 8, wherein the longitudinal centerline of the longitudinal lower cervical and thoracic supporting segment 7 is aligned respectively to the longitudinal centerlines of the transverse head and upper cervical supporting pillow 6 and the transverse lumbar supporting segment 8, forming a symmetrical assembly, as well as a left cut-out 18 and a right cut-out 19 next to the lower cervical and thoracic supporting segment 7 for placing the shoulder and arms, and for rolling from the supine to the lateral sleeping position.

An embodiment of the transverse head and upper cervical supporting pillow 6 is in a cuboid shape with a typical pillow dimension, wherein the top surface is recessed in both the transverse and longitudinal directions along the centerlines. The transverse center portion 10 and transverse lower portion 9 of the transverse head and upper cervical supporting pillow 6 are for supporting the head and upper cervical vertebra respectively. The transverse upper portion 11 of the transverse head and upper cervical supporting pillow 6 provides an alternative height and profile option to that of the transverse lower portion 9 in the transverse head and upper cervical supporting pillow 21 of another embodiment of the ergonomic back pillow assembly 20 with three discrete units as illustrated in

FIG. 7. The longitudinal center portion 13, left portion 12, and right portion 14 of the transverse head and upper cervical supporting pillow 6 are for supporting the head and upper cervical vertebra in the supine, left and right lateral sleeping positions respectively.

An embodiment of the longitudinal lower cervical and thoracic supporting segment 7 is roughly in a half cylindrical shape with a longitudinal length matching the lower cervical and thoracic span, and a top profile matching the back profile within the scapular distance and supporting the neutral cervical thoracic alignment when compressed under the body weight in the supine sleeping position. Its top and side profiles are in a gradual and smooth transition to provide sleep comfort and different levels of back support to a range of sleeping positions from the supine to the lateral sleep. For people with snoring problems, this significantly reduces the probability of supine sleep, minimizing the chance of heavy snoring and OSA. In the mean time, this could also help reducing the probability of brain diseases such as Alzheimer's disease.

An embodiment of the transverse lumbar supporting segment 8 is also roughly in a half cylindrical shape with a transverse length equal or slightly longer than that of the head and upper cervical supporting pillow 6, and with longitudinal center, left and right top profiles 16, 15 and 17 matching the lumbar pelvic span of the supine and lateral profiles respectively, and supporting the neutral lumbar pelvic alignment in the supine, left and right lateral sleeping positions respectively when compressed under the body weight.

The longitudinal center portions of the ergonomic back pillow 5, including the longitudinal center portion 13 of the transverse head and upper cervical supporting pillow 6, the entirety of the longitudinal lower cervical and thoracic supporting segment 7, and the longitudinal center portion 16 of the transverse lumbar supporting segment 8, form an ergonomic supine sleep entity for the supine sleeping position. The left cut-out 18 and right cut-out 19 next to the longitudinal lower cervical and thoracic supporting segment 7 are for placing the shoulder and arms, and for rolling from the supine to the lateral sleeping positions. The height and profiles of the ergonomic supine sleep entity allows the head to be elevated relative to the heart, a mandible-cervical angle for an open airway and minimal snoring, the shoulder and chest to be fully open, thus improving the respiration and cardiovascular functions.

The longitudinal left and right portions of the ergonomic back pillow 5, including the longitudinal left and right portions 12, 14 of the transverse head and upper cervical supporting pillow 6, and the longitudinal left and right portions 15, 17 of the transverse lumbar supporting segment 8, form an ergonomic lateral sleep entity for the lateral sleeping positions. The height and profiles of the ergonomic lateral sleep entity allows the head to be elevated relative to the heart for the ease of blood circulation, and the pressure on the shoulder and the hip to be reduced to the average pressure.

FIG. 7 shows a back view of an embodiment of the ergonomic back pillow assembly 20 composed of three discrete segments, including a transverse head and upper cervical supporting pillow 21, a longitudinal lower cervical and thoracic supporting segment 22, a transverse lumbar supporting segment 23, each with Velcro strips 24 on its bottom covers attached to the matching Velcro strips 26 on the top cover of a thin mat 25 in its front perspective view illustrated in FIG. 8. Together, the ergonomic back pillow assembly 20 and the thin mat 25 form an ergonomic back pillow pad assembly, adjustable in the longitudinal length by controlling the gap length between the adjacent discrete units, and in the height and profile by adding different number of layers of the stuffing materials with the same or different thickness under the top profile layer in each compartment of the discrete units, thus providing better tolerance and more flexibility to adapt to people with different height and body types, and to the corrective needs of people with various degrees of different types of musculoskeletal disorders.

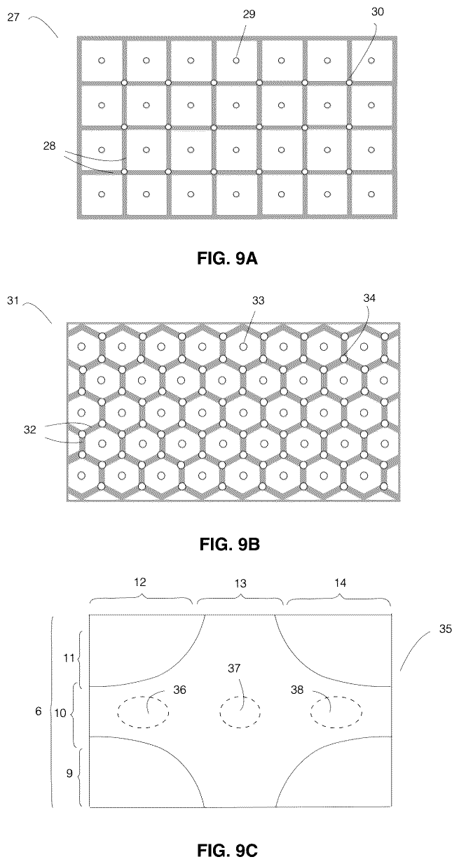

FIG. 9A to 9C illustrate the top views of three exemplary ventilation pattern 27, 31, and 35 of square patterned grooves with vertical through holes, honeycomb patterned grooves with vertical through holes, and three large vertical through voids respectively on a memory foam transverse head and upper cervical supporting pillow in an embodiment of the invented ergonomic back pillow. The ventilation pattern 27 has square patterned grooves 28 with vertical through hole 29 at the center of each foam square and vertical through hole 30 at the cross of the grooves. The ventilation pattern 31 has honeycomb patterned grooves 32 with vertical through hole 33 at the center of each foam honeycomb and vertical through hole 34 at the cross of the grooves. The cross-section views of the groove and vertical through hole can be in many geometric shapes. The ventilation pattern 35 has three large vertical through voids 37, 36, and 38 at the center of the supine, left and right lateral sleeping positions respectively. The large vertical through voids 36 and 38 also create space for the ears in the lateral sleep positions. The ventilation patterns can be in different geometric shapes other than the above, and can also be the combination of them.

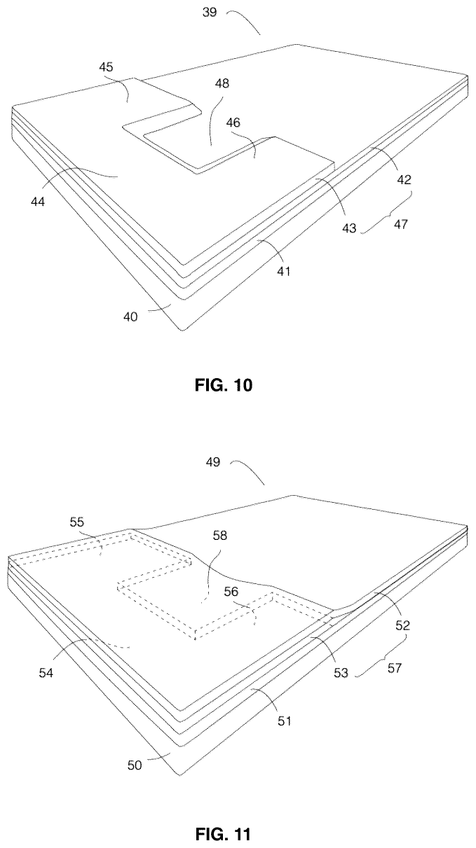

FIG. 10 shows a side perspective view of an embodiment of a multi-layer ergonomic leg elevation mattress assembly 39 with an ergonomic leg support stand alone unit 43 on the top surface. The ergonomic leg elevation mattress assembly 39, from the bottom to the top, is composed of a bottom layer 40 of innerspring firm support, a middle layer 41 of a medium firmness memory foam, a top layer 42 and an ergonomic leg support stand alone unit 43 both made of soft memory foam. The top layer 42 and ergonomic leg support stand alone unit 43 form an ergonomic leg elevation mattress 47. In the ergonomic leg support stand alone unit 43, its transverse lower portion 44 is for elevating the legs for better venous return when lying straight in the supine and lateral sleeping positions, its longitudinal upper left and upper right wings 45, 46 provide elevation and side supports for the legs to bend up sideway and anchor in the lateral sleeping positions, and its upper center cut-out 48 is for placing the hip and upper thighs. The size of the ergonomic leg support stand alone unit 43 is adaptive to the mattress standard, and it is adjustable in the height by stacking one or multiple layers of the ergonomic leg support stand alone unit 43 with the same or different thickness; in the position by moving the ergonomic leg support stand alone unit 43 along the longitudinal direction of the top layer 42 or extending the longitudinal upper left wing 45 and the longitudinal upper right wing 46 with fitting pads; and in the size and height of the cut-out 48 by filling the cut-out 48 with fitting pads of one layer or multiple layers with the same or different thickness along the longitudinal direction.

FIG. 11 shows a side perspective view of another embodiment of a multi-layer ergonomic leg elevation mattress assembly 49 with an ergonomic leg support stand alone unit 53, illustrated in the dash lines, inserted under the lower half of a top layer 52. The ergonomic leg elevation mattress assembly 49, from the bottom to the top, is composed of a bottom layer 50 of innerspring firm support, a middle layer 51 of a medium firmness memory foam, an ergonomic leg support stand alone unit 53 insert and a top layer 52 both made of soft memory foam. The top layer 52 and ergonomic leg support stand alone unit 53 insert form an ergonomic leg elevation mattress 57. In the ergonomic leg support stand alone unit 53, its transverse lower portion 54 is for supporting the legs for better venous return when lying straight in the supine and lateral sleeping positions, its longitudinal upper left and upper right wings 55, 56 provide elevation and side supports for the legs to bend up sideway and anchor in the lateral sleeping positions, and its upper center cut-out 58 is for placing the hip and upper thighs. Comparing to 39, the ergonomic leg elevation mattress assembly 49 offers the benefits of minimal topography change from that of a conventional mattress, a natural and gradual supporting profile under the leg compression, and a better structural stability. The size, height, and position of the ergonomic leg support stand alone unit 53, the size and height of the longitudinal upper left wing 55 and the longitudinal upper right wing 56, and the size and height of the cut-out 58 are adjustable in the same ways as described above.

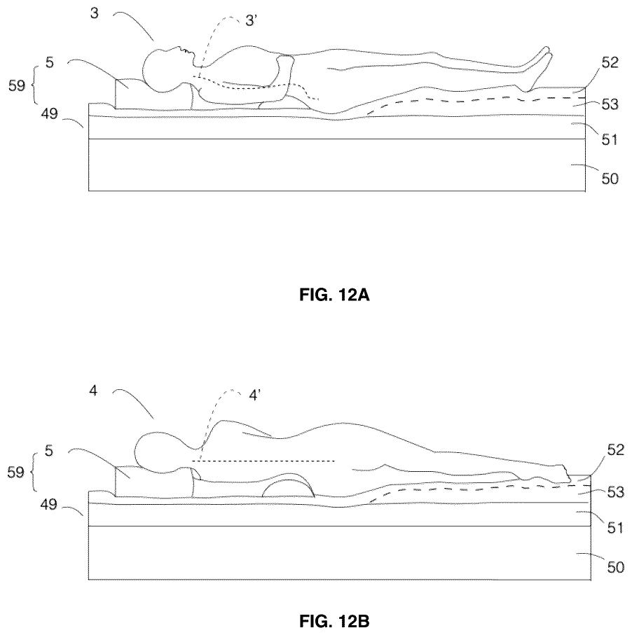

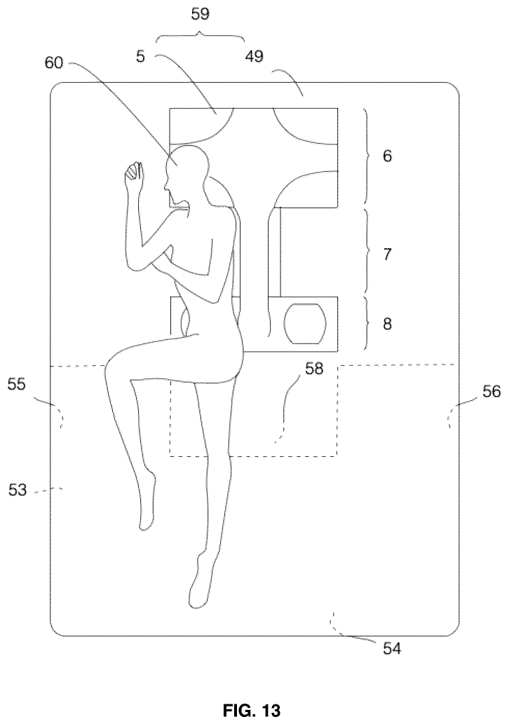

FIG. 12A and 12B show the side views of a human body 3 and 4 with a neutral spine alignment 3′ and 4′, illustrated in the dash lines, lying in the respective supine and lateral sleeping positions on an ergonomic full body sleep support system 59 composed of an ergonomic back pillow 5 and an ergonomic leg elevation mattress assembly 49 that, from the bottom to the top, is composed of a bottom layer 50 of innerspring firm support, a middle layer 51 of a medium firmness memory foam, an ergonomic leg support stand alone unit 53 insert and a top layer 52 both made of soft memory foam.

FIG. 13 shows a top view of a human body 60 with a neutral spine alignment lying in the lateral sleeping position with a leg bent up sideway and anchored on the upper left elevation wing 55 of an ergonomic full body sleep support system 59 composed of an ergonomic back pillow 5 and an ergonomic leg elevation mattress assembly 49 with an ergonomic leg support stand alone unit 53 insert, wherein its transverse lower portion 54 is for supporting the legs for better venous return when lying straight in the supine and lateral sleeping positions, its longitudinal upper left and upper right wings 55, 56 provide elevation and side supports for the legs to bend up sideway and anchor in the lateral sleeping positions, and its upper center cut-out 58 is for placing the hip and upper thighs. Comparing to anchoring the body linearly in the lateral sleeping position, having one leg bent up at various bending angles provides the body a more stable support on a triangular surface to help with the weight and heat distribution in the lateral and a range of sleeping positions between the supine and lateral sleeping positions.

The foregoing description is illustrative of particular embodiments of the invention, but is not meant to be a limitation upon the practice thereof. The following claims, including all equivalents thereof, are intended to define the scope of the invention.

What is claimed is:

1. An ergonomic back pillow to support a global neutral spinal pelvic alignment of the head, neck, torso, and hip:
comprising three segments of a transverse head and upper cervical supporting pillow, a longitudinal lower cervical and thoracic supporting segment, and a transverse lumbar supporting segment, wherein the longitudinal centerline of said longitudinal lower cervical and thoracic supporting segment is aligned to the longitudinal centerlines of said transverse head and upper cervical supporting pillow and said transverse lumbar supporting segment, forming a longitudinally symmetrical assembly; and
comprising a left cut-out and a right cut-out surrounded by said three segments for placing the shoulder and arms, and for rolling from the supine to the lateral sleeping position; and
wherein the longitudinal center portion of said transverse head and upper cervical supporting pillow, the entirety of said longitudinal lower cervical and thoracic supporting segment, and the longitudinal center portion of said transverse lumbar supporting segment, form an ergonomic supine sleep entity with dimension and profile, when compressed under the body weight, provide said neutral spinal pelvic alignment, an elevated head relative to the heart, a mandible-cervical angle for open airway and minimal snoring, an open shoulder and chest, and a uniform contact pressure on the body parts in the supine sleeping position; and
wherein the longitudinal left and right portions of said transverse head and upper cervical supporting pillow, and the longitudinal left and right portions of said transverse lumbar supporting segment, form an ergonomic lateral sleep entity with dimension and profile, when compressed under the body weight, provide said neutral spinal pelvic alignment, an elevated head relative to the heart, an open shoulder and chest, and a uniform contact pressure on the body parts in the lateral sleeping positions; and
wherein the top and side profiles of said ergonomic back pillow has a smooth, gradual and natural transition from said supine sleep entity to said lateral sleep entity, supporting a range of sleeping positions from the supine to the lateral sleep.

2. An ergonomic back pillow of claim 1, is in the form of:
a single unit containing said three segments; or
discrete units containing one or two of said three segments; and
wherein said single unit or said discrete units are enclosed in a fabric cover forming an ergonomic back pillow unit; and
wherein said discrete units are attached to each other by fabric fasteners forming an ergonomic back pillow assembly, or attached to a thin mat by fabric fasteners forming an ergonomic back pillow pad assembly; and
wherein said single unit and each unit of said discrete units are further divided into compartments; and
wherein said compartment of said single unit or said discrete units have one layer or multiple layers of the stuffing materials; and
wherein said ergonomic back pillow is in a single-form or a side-by-side fused plurality-form.

3. An ergonomic back pillow of claim 2, is adjustable:
in the longitudinal length by controlling the gap length between the adjacent said discrete units; and
in the height and profile by adding different number of layers of the stuffing materials with the same or different thickness under the top profile layer in said compartment of said single unit or said discrete units; and
in the two alternative head and upper cervical top profile layers provided by the different upper and lower transverse halves of said transverse head and upper cervical supporting pillow for people with different preference.

4. An ergonomic back pillow of claim 2, wherein each said layer of said compartment and said thin mat contain the same or different stuffing materials, said stuffing materials comprising synthetic materials, plant based materials, animal-based materials, metal containing materials or any of their combinations.

5. An ergonomic back pillow of claim 4, wherein said stuffing materials is enclosed in a fabric cover, said fabric cover:
is in the form of said single unit or said discrete units with said compartments; and
is composed of the same or different materials of plant-based fiber, animal-based fiber, synthetic fiber, metal fiber, metal containing fiber, metal infused fiber, or any of their combinations; and
contains features for the opening and cleaning.

6. An ergonomic back pillow of claim 4 or 5, wherein:
said synthetic materials include foam, shredded foam, 3D air-weave polymer fiber network, latex, and synthetic fibers; and
said plant based materials include cotton, kapok, flax, hemp, ramie, jute, sisal, coir, reed, luffa sponge, Rayon, Modal, Viscose, Lyocell, buckwheat hull, millet hull, dried leaves, and straws; and
said animal-based materials include wool, silk, down, and feather; and
said metal containing materials include metal fiber or weave, metal coated or plated fiber or weave, metal ribbon or metal coated ribbon wrapped fiber or weave, metal coated or metal, plated 3D structures of luffa or reticulated foam or air-weave polymer fiber network, metal oxide or metal salt infused fiber or weave, and metal or metal oxide or metal salt infiltrated foam; or
any of their combinations; and
wherein additional features are built into said foam and said metal or metal oxide or metal salt infiltrated foam for better thermal and moisture management and air permeability, including arrays of vertical through-holes, grooves and/or grids on the foam surfaces and/or the foam internal, large vertical through voids, and any of their combinations.

7. An ergonomic leg support to elevate the legs and facilitate their venous return, comprising:
a transverse lower portion to elevate the legs when lying straight in the supine and lateral sleeping positions; and
a longitudinal upper left wing and a longitudinal upper right wing above said transverse lower portion for the elevation and side support of the legs to bend up sideway and anchor in the lateral sleeping positions; and
a cut-out surrounded by said transverse lower portion, said longitudinal upper left wing and said longitudinal upper right wing for placing the hip and upper thighs.

8. An ergonomic leg support of claim 7, is in the form of:
a stand alone unit comprising one layer or multiple layers of the stuffing materials with the same or different thickness; or
a part of an ergonomic leg elevation mattress comprising or merging said stand alone unit on the top or at the bottom of a mattress; or
a part of an ergonomic leg elevation mattress assembly comprising at least a bottom support layer and said ergonomic leg elevation mattress; and
wherein said ergonomic leg support is in a single-form or a side-by-side fused plurality-form.

9. An ergonomic leg support of claim 8, is adjustable:
in the elevation height by stacking one layer or multiple layers of said stand alone unit with the same or different thickness; and
in the position by moving said stand alone unit along the longitudinal direction of said mattress in said ergonomic leg elevation mattress or extending said longitudinal upper left wing and said longitudinal upper right wing with fitting pads; and
in the size and height of said cut-out by filling with fitting pads in the longitudinal direction.

10. An ergonomic leg support of claim 9, wherein each said layer of said stand alone unit, said mattress of said ergonomic leg elevation mattress, said bottom support layer of said ergonomic leg elevation mattress assembly, and said fitting pad contain the same or different stuffing materials, said stuffing materials comprising innerspring, synthetic materials, plant based materials, animal-based materials, metal containing materials, or any of their combination.

11. An ergonomic leg support of claim 10, wherein said stuffing materials is enclosed in a fabric cover, said fabric cover:
is in the form of a single piece with multiple compartments for the ease of the adjustment of said transverse lower portion, said longitudinal upper left wing, said longitudinal upper right wing, and said fitting pad extensions; and
is composed of the same or different materials of plant-based fiber, animal-based fiber, synthetic fiber, metal fiber, metal containing fiber, metal infused fiber, or any of their combinations; and
contains features for the opening and cleaning.

12. An ergonomic leg support of claim 10 or 11, wherein:
said synthetic materials include foam, shredded foam, 3D air-weave polymer fiber network, latex, and synthetic fibers; and
said plant based materials include cotton, kapok, flax, hemp, ramie, jute, sisal, coir, reed, luffa sponge, Rayon, Modal, Viscose, and Lyocell; and
said animal-based materials includes animal hair; and
said metal containing materials include metal coated or plated 3D structures, and metal or metal oxide or metal salt infiltrated foam; or
any of their combination; and
wherein additional features are built into said foam and said metal or metal oxide or metal salt infiltrated foam for better thermal and moisture management and air permeability, including arrays of vertical through-holes, grooves and/or grids on the foam surfaces and/or the foam internal, or any of their combinations.

13. An ergonomic full body sleep support system for the global neutral musculoskeletal alignment, in a single-form or a side-by-side fused plurality-form, wherein:
said ergonomic back pillow to support a global neutral spinal pelvic alignment of the head, neck, torso, and hip:
comprising three segments of a transverse head and upper cervical supporting pillow, a longitudinal lower cervical and thoracic supporting segment, and a transverse lumbar supporting segment, wherein the longitudinal centerline of said longitudinal lower cervical and thoracic supporting segment is aligned to the longitudinal centerlines of said transverse head and upper cervical supporting pillow and said transverse lumbar supporting segment, forming a longitudinally symmetrical assembly; and
comprising a left cut-out and a right cut-out surrounded by said three segments for placing the shoulder and arms, and for rolling from the supine to the lateral sleeping position; and
wherein the longitudinal center portion of said transverse head and upper cervical supporting pillow, the entirety of said longitudinal lower cervical and thoracic supporting segment, and the longitudinal center portion of said transverse lumbar supporting segment, form an ergonomic supine sleep entity with dimension and profile, when compressed under the body weight, provide said neutral spinal pelvic alignment, an elevated head relative to the heart, a mandible-cervical angle for open airway and minimal snoring, an open shoulder and chest, and a uniform contact pressure on the body parts in the supine sleeping position; and
wherein the longitudinal left and right portions of said transverse head and upper cervical supporting pillow, and the longitudinal left and right portions of said transverse lumbar supporting segment, form an ergonomic lateral sleep entity with dimension and profile, when compressed under the body weight, provide said neutral spinal pelvic alignment, an elevated head relative to the heart, an open shoulder and chest, and a uniform contact pressure on the body parts in the lateral sleeping positions; and
wherein the top and side profiles of said ergonomic back pillow has a smooth, gradual and natural transition from said supine sleep entity to said lateral sleep entity, supporting a range of sleeping positions from the supine to the lateral sleep; and
said ergonomic leg support to elevate the legs and facilitate their venous return, comprising:
a transverse lower portion to elevate the legs when lying straight in the supine and lateral sleeping positions; and
a longitudinal upper left wing and a longitudinal upper right wing above said transverse lower portion for the elevation and side support of the legs to bend up sideway and anchor in the lateral sleeping positions; and
a cut-out surrounded by said transverse lower portion, said longitudinal upper left wing and said longitudinal upper right wing for placing the hip and upper thighs.

14. An ergonomic full body sleep support system of claim 13, wherein:
said ergonomic back pillow, is in the form of:
a single unit containing said three segments; or
discrete units containing one or two of said three segments; and
wherein said single unit or said discrete units are enclosed in a fabric cover forming an ergonomic back pillow unit; and
wherein said discrete units are attached to each other by fabric fasteners forming an ergonomic back pillow assembly, or attached to a thin mat by fabric fasteners forming an ergonomic back pillow pad assembly; and
wherein said single unit and each unit of said discrete units are further divided into compartments; and
wherein said compartment of said single unit or said discrete units have one layer or multiple layers of the stuffing materials.
wherein said ergonomic back pillow is in a single-form or a side-by-side fused plurality-form; and
said ergonomic leg support, is in the form of:
a stand alone unit comprising one layer or multiple layers of the stuffing materials with the same or different thickness; or
a part of an ergonomic leg elevation mattress comprising or merging said stand alone unit on the top or at the bottom of a mattress; or
a part of an ergonomic leg elevation mattress assembly comprising at least a bottom support layer and said ergonomic leg elevation mattress; and
wherein said ergonomic leg support is in a single-form or a side-by-side fused plurality-form.

15. An ergonomic full body sleep support system of claim 14, wherein:
said ergonomic back pillow, is adjustable:
in the longitudinal length by controlling the gap length between the adjacent said discrete units; and
in the height and profile by adding different number of layers of the stuffing materials with the same or different thickness under the top profile layer in said compartment of said single unit or said discrete units; and
in the two alternative head and upper cervical top profile layers provided by the different upper and lower transverse halves of said transverse head and upper cervical supporting pillow for people with different preference; and
said ergonomic leg support, is adjustable:
in the elevation height by stacking one layer or multiple layers of said stand alone unit with the same or different thickness; and
in the position by moving said stand alone unit along the longitudinal direction of said mattress in said ergonomic leg elevation mattress or extending said longitudinal upper left wing and said longitudinal upper right wing with fitting pads; and
in the size and height of said cut-out by filling with fitting pads in the longitudinal direction.

16. An ergonomic full body sleep support system of claim 14, wherein:
said ergonomic back pillow, wherein each said layer of said compartment and said thin mat contain the same or different stuffing materials, said stuffing materials comprising synthetic materials, plant based materials, animal-based materials, metal containing materials or any of their combinations:
wherein said synthetic materials include foam, shredded foam, 3D air-weave polymer fiber network, latex, and synthetic fibers; and
wherein said plant based materials include cotton, kapok, flax, hemp, ramie, jute, sisal, coir, reed, luffa sponge, Rayon, Modal, Viscose, Lyocell, buckwheat hull, millet hull, dried leaves, and straws; and
wherein said animal-based materials include wool, silk, down, and feather; and
wherein said metal containing materials include metal fiber or weave, metal coated or plated fiber or weave, metal ribbon or metal coated ribbon wrapped fiber or weave, metal coated or metal, plated 3D structures of luffa or reticulated foam or air-weave polymer fiber network, metal oxide or metal salt infused fiber or weave, and metal or metal oxide or metal salt infiltrated foam; or
wherein additional features are built into said foam and said metal or metal oxide or metal salt infiltrated foam for better thermal and moisture management and air permeability, including arrays of vertical through-holes, grooves and/or grids on the foam surfaces and/or the foam internal, large vertical through voids, and any of their combinations; and
said ergonomic leg support, wherein each said layer of said stand alone unit, said mattress of said ergonomic leg elevation mattress, said bottom support layer of said ergonomic leg elevation mattress assembly, and said fitting pad contain the same or different stuffing materials, said stuffing materials comprising innerspring, synthetic materials, plant based materials, animal-based materials, metal containing materials, or any of their combination:
wherein said synthetic materials include foam, shredded foam, 3D air-weave polymer fiber network, latex, and synthetic fibers; and
wherein said plant based materials include cotton, kapok, flax, hemp, ramie, jute, sisal, coir, reed, luffa sponge, Rayon, Modal, Viscose, and Lyocell; and
wherein said animal-based materials includes animal hair; and
wherein said metal containing materials include metal coated or plated 3D structures, and metal or metal oxide or metal salt infiltrated foam; or
wherein additional features are built into said foam and said metal or metal oxide or metal salt infiltrated foam for better thermal and moisture management and air permeability, including arrays of vertical through-holes, grooves and/or grids on the foam surfaces and/or the foam internal, or any of their combinations.

17. An ergonomic full body sleep support system of claim 14, wherein:
said ergonomic back pillow, wherein said stuffing materials is enclosed in a fabric cover, said fabric cover:
is in the form of said single unit or said discrete units with said compartments; and
is composed of the same or different materials of plant-based fiber, animal-based fiber, synthetic fiber, metal fiber, metal containing fiber, metal infused fiber, or any of their combinations; and
contains features for the opening and cleaning; and
said ergonomic leg support, wherein said stuffing materials is enclosed in a fabric cover, said fabric cover:
is in the form of a single piece with multiple compartments for the ease of the adjustment of said transverse lower portion, said longitudinal upper left wing, said longitudinal upper right wing, and said fitting pad extensions; and
is composed of the same or different materials of plant-based fiber, animal-based fiber, synthetic fiber, metal fiber, metal containing fiber, metal infused fiber, or any of their combinations; and
contains features for the opening and cleaning.
